(19) United States
(12) Patent Application Publication
(10) Pub. No.: US 2012/0303055 A1
(43) Pub. Date: Nov. 29, 2012

(54) PERCUTANEOUS ROD DELIVERY TECHNIQUES AND SYSTEMS

(75) Inventors: Gregory C. Marik, Collierville, TN (US); Newton H. Metcalf, JR., Memphis, TN (US)

(73) Assignee: Warsaw Orthopedic, Inc., Warsaw, IN (US)

(21) Appl. No.: 13/117,362

(22) Filed: May 27, 2011

(51) Int. Cl. A61B 17/00

(52) U.S. Cl. 606/205

(57) ABSTRACT

There are disclosed techniques for positioning a connecting element adjacent one or more bones or bony portions, such as the spinal column, through a minimally invasive surgical approach. The system generally includes at least one bone anchor engageable to the one or more bones or bony portions and at least one extender removably engaged to the bone anchor. A connecting element is movable into the patient and guided by the at least one extender to a first position in the patient. A second bone anchor and extender is then inserted into the patient and engaged to a bony portion. The connecting element is then moved from the at least one extender to extend to the second bone anchor and second extender. The connecting element is then engaged to each of the bone anchors.

### BACKGROUND

Various devices and methods for stabilizing bone structures have been used for many years. For example, the fracture of an elongated bone, such as a femur or humerus, can be stabilized by securing a plate to the fractured bone across the fracture. The plate extends across the fractured area and thus stabilizes the fractured components of the bones relative to one another in a desired position. When the fracture heals, the plate can be removed or left in place, depending on the type of plate that is used.

Another type of stabilization technique uses one or more elongated rods extending between components of a bony structure and secured to the bony structure to stabilize the components relative to one another. The components of the bony structure are exposed and one or more bone engaging fasteners are placed into each component. The elongated rod is then secured to the bone engaging fasteners in order to stabilize the components of the bony structure.

One problem associated with the above described stabilization structures is that the skin and tissue surrounding the surgical site must be cut, removed, and/or repositioned in order for the surgeon to access the location where the stabilization device is to be installed. This repositioning of tissue causes trauma, damage, and scarring to the tissue. There are also risks that the tissue will become infected and that a long recovery time will be required after surgery for the tissue to heal.

Minimally invasive surgical techniques are particularly desirable in, for example, spinal and neurosurgical applications because of the need for access to locations deep within the body and the presence of vital intervening tissues. The development of percutaneous minimally invasive spinal procedures has yielded a major improvement in reducing recovery time and post-operative pain because they require minimal, if any, muscle dissection and can be performed under local anesthesia. These benefits of minimally invasive techniques have also found application in surgeries for other locations in the body where it is desirable to minimize tissue disruption and trauma. However, there remains a need for further improvements in instruments, systems and methods for stabilizing bony structures using minimally invasive techniques.

### SUMMARY

One nonlimiting embodiment of the present application is directed to a technique for positioning a connecting element adjacent one or more bones or bony portions, such as the spinal column, through a minimally invasive surgical approach. The system generally includes at least one first bone anchor engageable to the one or more bones or bony portions and at least one first elongated extender removably engaged to the first bone anchor. A connecting element inserter instrument is engageable with an elongated connecting element to move the connecting element into the patient to the first bone anchor or the first extender. After the connecting element is moved into the patient, a second anchor with a second elongated extender removably engaged to the second bone anchor is positioned into the patient and engaged to bony structure. The connecting element is then manipulated for positioning into the second anchor extender or the second bone anchor while remaining positioned in the first bone anchor or first anchor extender. However, in other embodiments, different forms and applications are envisioned.

For example, another embodiment of the subject application is directed to a system for minimally invasive surgery that includes at least one bone anchor including a distal bone engaging portion and a proximal receiving portion. The system also includes at least one elongated extender extending along a longitudinal axis between a proximal end portion and a distal end portion configured to releasably engage with the at least one bone anchor. An elongated connecting element is inserted in the patient and positioned in the first anchor extender. A second bone anchor is then engaged to a second bony structure by referencing the connecting element in the patient. The inserted connecting element is then guided by the at least one extender into each of the first and second bone anchors for implantation in the patient.

In one embodiment, the at least one extender includes a passage extending between its proximal and distal end portions. The elongated connecting element is positionable through the passage of the at least one extender and is movable along the longitudinal axis of the at least one extender from the proximal end portion toward the distal end portion. After the connecting element is positioned in the passage of the at least one extender, a second anchor and second elongated extender are engaged to bony structure in the patient. The connecting element is then moved from the passage of the at least one extender to the second anchor extender, and then guided along the extenders into receiving portions of the bone anchors.

In yet another embodiment, the connecting element is positioned in the patient and is located in the passage of the at least one extender so that the connecting element projects outwardly from the at least one extender. The connecting element is movable transversely to the at least one extender to a first position that is references for insertion of a second anchor and second elongated extender into the patient. The connecting element is then moved transversely to the at least one extender to the second extender so that the connecting element extends between the extenders. The connecting element is then moved distally along the extenders to the bone anchors engaged to the extenders. The connecting element can then be engaged to the bone anchors and the elongated extenders are removed from the bone anchors to provide a low profile construct for implantation in the patient.

Another embodiment of the present application is a unique system for minimally invasive surgery in a patient. Other embodiments include unique methods, systems, devices, kits, assemblies, equipment, and/or apparatus involving minimally invasive surgical systems and techniques.

Further embodiments, forms, features, aspects, benefits, objects and advantages of the present application shall become apparent from the detailed description and figures provided herewith.

### BRIEF DESCRIPTION OF THE FIGURES

FIG. 1 is a perspective of a stabilization system for a spinal column segment.

FIG. 2 is a diagrammatic side elevation view of a spinal column segment and a first step for positioning a connecting element in a patient in a minimally invasive surgical procedure.

FIG. 3 is a diagrammatic side elevation view of the spinal column segment of FIG. 2 and a second step for positioning the connecting element in the patient in the minimally invasive surgical procedure.

FIG. 4 is a diagrammatic side elevation view of the spinal column segment of FIG. 2 and a third step for positioning the connecting element in the patient in the minimally invasive surgical procedure.

FIG. 5 is a diagrammatic side elevation view of the spinal column segment of FIG. 2 and a fourth step for positioning the connecting element in the patient in the minimally invasive surgical procedure.

FIG. 6 is a diagrammatic side elevation view of a spinal column segment and a first step for positioning a connecting element in a patient in another embodiment minimally invasive surgical procedure.

FIG. 7 is a diagrammatic side elevation view of a spinal column segment of FIG. 6 and a second step for positioning the connecting element in the patient in the minimally invasive surgical procedure.

FIG. 8 is a diagrammatic side elevation view of a spinal column segment of FIG. 6 and a third step for positioning the connecting element in the patient in the minimally invasive surgical procedure.

FIG. 9 is a diagrammatic side elevation view of a spinal column segment of FIG. 6 and a fourth step for positioning the connecting element in the patient in the minimally invasive surgical procedure.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

The subject application is generally directed to systems and techniques for positioning a connecting element adjacent one or more bones or bony portions, such as the spinal column, through a minimally invasive surgical approach. The systems generally include a number of bone anchors engageable to the one or more bones or bony portions and a number of anchor extenders removably engaged to the bone anchors. At least one of the bone anchors is engaged to a bony portion with an extender extending from a bone engaging portion of the bone anchor. A connecting element is positionable into the at least one anchor extender in the patient, and is movable relative to the at least one anchor extender to allow subsequent engagement of a second bone anchor and second anchor extender to another bony portion. The connecting element can provide a reference for the desired positioning and orientation of the second anchor and second anchor extender. The connecting element is then movable from the first position into a second position in which the connecting element extends between and connects the bone anchors to one another. The connecting element can then be secured to the bone anchors to provide stabilization of the bony portions, and the extenders are removed from the patient. When moving from the first position to the second position, the connecting element is moved subcutaneously. It is contemplated that the extenders extend from the bone anchors and project from the skin of the patient to provide a visual reference of the bone anchor location in the patient. An incision may be made through the skin and tissue that extends from one extender to the other to provide a pathway for movement of an inserter connected to the connecting element. However, it is also contemplated that the extenders extend through respective punctures in the skin and the connecting element is moved subcutaneously from the first position to the second position by manipulating the connecting element through one of the extenders, or through a small incision that extends only part of the way between the extenders.

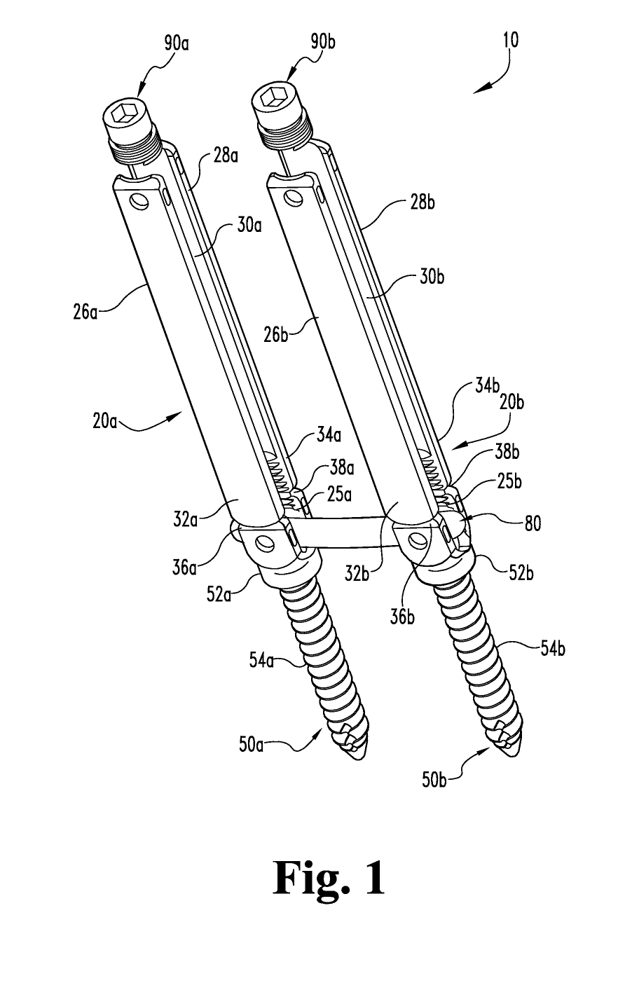

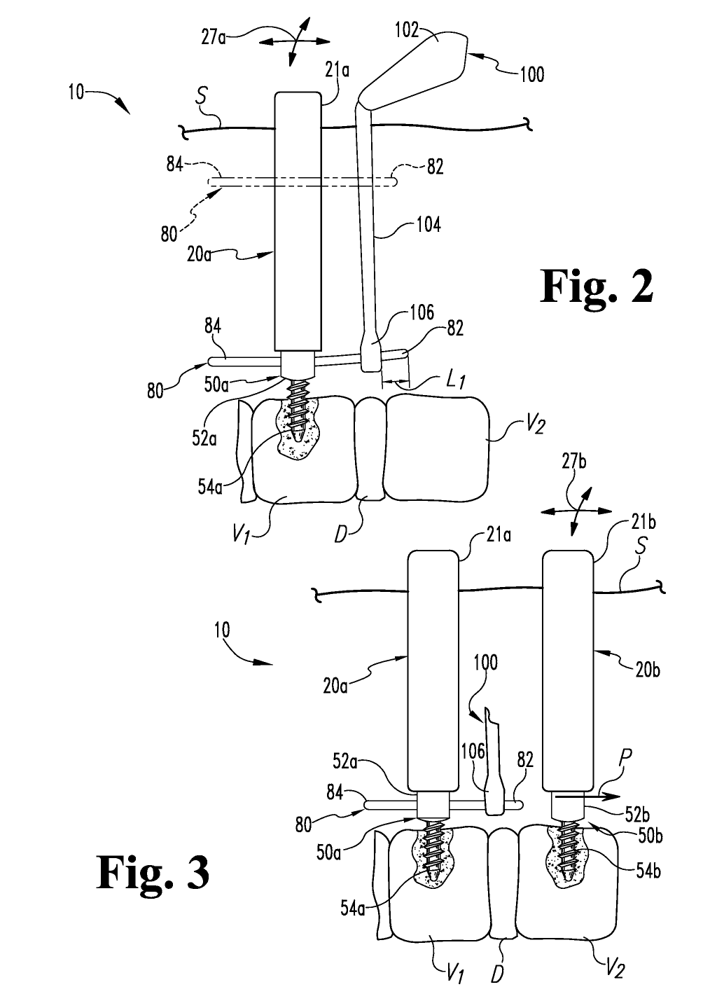

Referring now to FIG. 1, there is shown a minimally invasive surgical system 10 that is positionable relative to a portion of the spinal column including adjacent vertebrae V₁, V₂ and a disc D (FIGS. 2-9) positioned therebetween. It should be appreciated that use of system 10 in connection with more than two adjacent vertebrae or even at other anatomical locations besides the spinal column are also contemplated. System 10 includes two anchor extenders 20a, 20b releasably mountable to respective ones of anchors 50a, 50b and a connecting element 80. An inserter instrument 100 may also be provided to hold and position connecting element 80 during insertion and implantation. In other non-illustrated forms, system 10 may include one or more anchors and/or anchor extenders in addition to anchors 50a, 50b and anchor extenders 20a, 20b for a multi-level stabilization procedure.

Anchors 50a, 50b include proximal receiving portions 52a, 52b configured to receive connecting element 80 and distal bone engaging portions 54a, 54b. Proximal receiving portions 52a, 52b are pivotally and rotatable mounted to distal bone engaging portions 54a, 54b. Other forms for anchors 50a, 50b are contemplated, including uni-axial and uni-planar forms. In the illustrated embodiment, bone engaging portions 54a, 54b are bone screws with a threaded shank to engage the bony structure of the underlying vertebrae V₁, V₂. The bone engaging portions 54a, 54b can also be in the form of a spike, staple, hook, fusion device, cannulated screw, fenestrated screw, interbody device, intrabody device, clamp, plate, suture anchor, bolt, pin or other bone engaging member. The receiving portions 52a, 52b can be in the form of a U-shaped saddle, yoke, eye-bolt or through-hole, side opening member, bottom opening member, top-opening member, eyelet, or any other structure engageable to connecting element 80. In the illustrated embodiment, each bone engaging portion 54a, 54b is a bone screw and each receiving portion 52, 52b is a saddle or U-shaped head pivotally mounted to the head of the bone screw, although it is also contemplated that anchors 50a, 50b can have different forms. Receiving portion 52a, 52b is movable to rotate around the head of the bone screw to align the side openings of passages of the receiving portions 52a, 52b to receive connecting element 80. The passages of receiving portions 52a, 52b also open proximally to receive connecting element 80 from a top-down approach, and also to receiver a set screw, cap or other engaging member to secure connecting element 80 in receiving portions 52a, 52b.

In the illustrated embodiment, proximal receiving portions 52a, 52b are receivers having a pair of opposing arms defining side-opening passages 25a, 25b to receive connecting element 80 in its implanted position shown in FIG. 1. The arms of the receiving portions 52a, 52b further define a proximally/distally extending opening that opens at a proximal end of the arms to receive a respective one of engaging members 90a, 90b to secure connecting element 80 in the passages of receiving portions 52a, 52b. Bone engaging portions 54a, 54b can be pivotally received in proximal receiving portions 52a, 52b through the distal openings thereof, and structured to interact therewith to provide anchors 50a, 50b with multi-axial capabilities that permit either a selected number of positions or infinitely numbered of positions of bone engaging portions 54a, 54b relative to proximal receiving portions 52a, 52b.

Each of extenders 20a, 20b includes elongated first and second tabs 26a, 28a and tabs 26b, 28b, respectively. Tabs 26a, 28a extend proximally from the arms of receiving portion 52a and define an elongated passage 30a that is in communication with passage 25a of receiving portion 52a at the proximal end of receiving portion 52a. Tabs 26b, 28b extend proximally from the arms of receiving portion 52b and define an elongated passage 30b that is in communication with passage 25b of receiving portion 52b at the proximal end of receiving portion 52b. Passages 30a, 30b define a path to the respective receiving portion 52a, 52b that extends from the skin of the patient to the implantation location defined by receiving portions 52a, 52b when bone engaging portions 54a, 54b are engaged to underlying bony structure. In the illustrated embodiment, passages 30a, 30b extend along the entire length of the respective extender 20a, 20b and open at both sides of each extender 20a, 20b. Passages 30a, 30b can also be configured to open at the opposite sides along only a portion of the length of the respective extender 20a, 20b.

The distal end portions 32a, 34a of tabs 26a, 28a can be internally threaded to receive engaging member 90a therein and to allow engaging member 90a to threadingly pass therethrough to threadingly engage internal threads in receiving portion 52a. The distal end portions 32b, 34b of tabs 26b, 28b can be internally threaded to receive engaging member 90b therein and to allow engaging member 90b to threadingly pass therethrough to threadingly engage internal threads in receiving portion 52b. The remaining portions of tabs 26a, 28a and tabs 26b, 28b can be non-threaded as shown, or threaded along all or a portion of the length thereof. Distal end portions 32a, 34a are removably engaged to receiving portion 52a by reduced strength portions 36a, 38a, respectively, that allows tabs 26a, 28a to be removed by twisting or torquing tabs 26a, 28a to sever it from receiving portion 52a at reduced strength portions 36a, 38a. Distal end portions 32b, 34b are removably engaged to receiving portion 52b by reduced strength portions 36b, 38b, respectively, that allows tabs 26b, 28b to be removed by twisting or torquing tabs 26b, 28b to sever it at reduced strength portions 36b, 38b. The reduced strength portions 36a, 36b, 38a, 38b can be formed by reducing the wall thickness of the tabs at their junction with the receiver, by providing perforations through the tab-receiving portion junction, or by any suitable removably joining means. Further details of one example of suitable anchors with removable extension portions are provided in U.S. Patent App. Pub. No. 2007/0191840 published on Aug. 16, 2007, which is incorporated herein by reference in its entirety. Other embodiments contemplate a suitable anchor and extender extending from the anchor for use in system 10, including anchors that do not include integral tabs but are provided with removably mounted extenders that clamp or are otherwise secured to receiving portions of the anchors.

Referring now to FIGS. 2-5, there is shown stabilization system 10 that includes a first anchor 50a and a second anchor 50b spaced from one another and engaged to underlying vertebrae V₁, V₂. Extenders 20a, 20b extend from the corresponding receiving portion 52a, 52b and are sized to extend proximally from the bone engaging portion 52a, 52b to a proximal ends 21a, 21b located through skin S and outside the patient to provide a path to receiving portion 52a, 52b in the patient. In FIGS. 2-9, vertebrae V₁, V₂ are shown diagrammatically and positioned below skin level S with tissue of the patient between the vertebrae V₁, V₂ and skin S. It is contemplated that the spinal column segment is part of a patient in which spinal surgery is to be performed with the present invention. It is also contemplated that the spinal column segment may comprise a non-human or non-living animal substrate, such as may be present with a training model to teach methods employing the surgical instruments and implants discussed herein.

Referring to FIG. 2, system 10 further includes an inserter 100 with a handle 102, an elongated shaft 104 extending distally from handle 102, and a distal grasping portion 106 configured to removably hold connecting element 80. Connecting element 80 is removably engageable to grasping portion 106 so that connecting element extends transversely to shaft 102 and transversely to and protruding from both sides of extender 20a. A first end 82 of connecting element 80 protrudes from grasping portion 106 with a length L1 sized to correspond at least to the width of receiving portion 52b of anchor 50b. The connecting element 80 is referenced to extender 20a by, for example, placing second end 84 through extender 20a in an initial insertion position, as shown in dashed lines in FIG. 2, and then advanced distally along passage 30a in the transverse orientation and guided by tabs 26a, 28a to or adjacent to receiving portion 52a. Extender 20a can be pivoted relative to anchor 50a to a desired orientation, as indicated by arrowed lines 27a, either before or after connecting element 80 is inserted into the patient. In addition, other referencing arrangements between connecting element 80 and extender 20a are also contemplated. For example, the inserter could be mounted to proximal end 21a of extender 20a and movable relative thereto to guide the connecting element to receiving portion 52a. In another example, the inserter could be manipulated from a position that will not be between the extenders 20a, 20b, but rather is initiated from a cephalad or caudal approach when the initial extender 20a is positioned cephalad or caudal, respectively, of the subsequent extenders 20b.

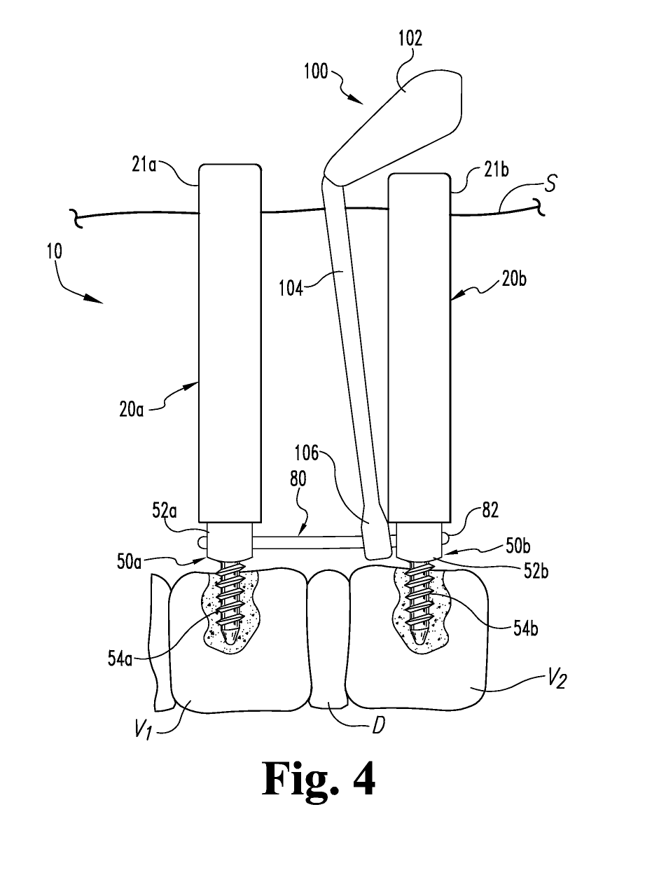

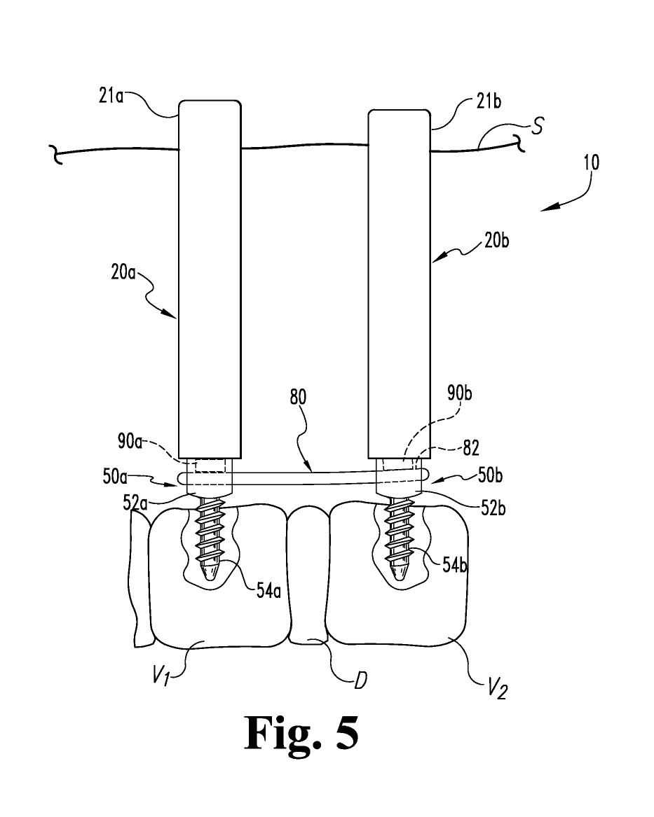

While connecting element 80 is held in the patient with inserter 100, a second anchor 50b and extender 20b are inserted into the patient and engaged to vertebra V₂, as shown in FIG. 3. The prior insertion and orienting of connecting element 80 and extender 20a allows connecting element 80 to be positioned to the first position along an initial insertion path that provides the least resistance. The connecting element 80 in its first position provides an indication of a desirable implantation location and orientation for anchor 50b and extender 20b to receive connecting element 80. For example, extender 20b can be pivoted universally about bone engaging portion 54b, such as indicated by arrowed lines 27b, to any parallel or non-parallel orientation relative to extender 20a. As shown in FIG. 4, when anchor 50b and extender 20b are positioned in the desired implantation location and orientation, first end portion 82 of connecting element 80 can be moved into receiving portion 52b of anchor 50b with inserter 100. Since length L1 corresponds to the width of receiving portion 52b, the surgeon is given an indication that connecting element 80 is properly positioned in receiving portion 52b when grasping portion 106 contacts receiving portion 52b. Insertion and manipulation of connecting element 80 may also be monitored fluoroscopically or with any suitable viewing system or technique. Connecting element 80 is movable with inserter 100 along insertion path P through tissue of the patient from a location outside the receiving portion 52b and then through receiving portion 52b so that connecting element is located in each of the receiving portions 52a, 52b. As shown in FIG. 5, engaging members 90a, 90b can then be guided along extenders 20a, 20b and into receiving portions 52a, 52b to secure connecting element 80 therein and inserter 100 is removed from the patient. Extenders 20a, 20b can then be removed from receiving portions 52a, 52b by severing tabs 26a, 28a and tabs 26b, 28b to provide a low profile stabilization construct in the patient.

It is also contemplated that after positioning connecting element 80 in receiving portion 52b, the leading end portion of connecting element 80 can provide a reference for insertion of a third anchor and third anchor extension for engagement to a third vertebra in a multi-level stabilization procedure. Connecting element 80 is then guided along path P to the receiving portion of the third anchor and then secured thereto with another engaging member to provide multi-level stabilization along with anchors 50a, 50b secured to connecting element 80.

It is contemplated that an incision can be made from extender 20a to extender 20b to accommodate movement of inserter 100 as it guides connecting element 80 to its implantation location in receiving portions 52a, 52b. Alternatively, a small incision can extend part of the way from extender 20a toward extender 20b, and inserter 100 manipulated through the small incision by pivoting grasping portion 106 toward receiving portion 52b below the skin and tissue to position first end 82 of connecting element 80 into receiving portion 52b while second end 84 of connecting element 80 is located in receiving portion 52a.

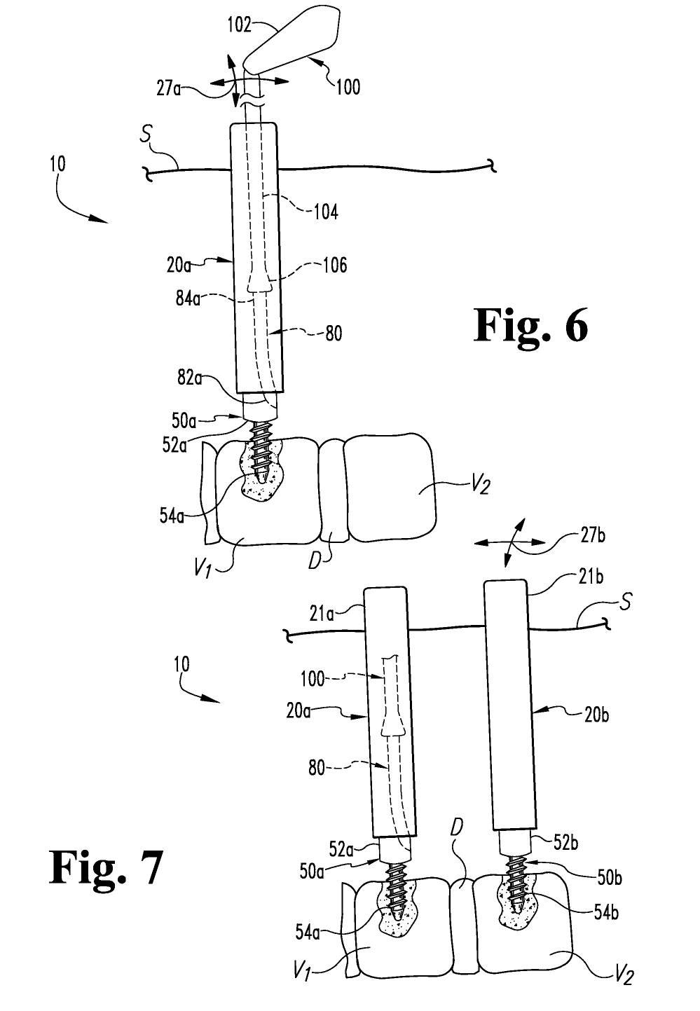

Referring to FIGS. 6-9, another embodiment insertion technique is shown in which connecting element 80 is guided by first extender 20a to a first position in the patient with inserter 100, as shown in FIG. 6. First end 82 of connecting element 80 is located at or adjacent to receiving portion 52a of anchor 50. The opposite second end 84 of connecting element 80 is grasped by inserter 100 so connecting element 80 can be placed through extender 20a in an initial insertion orientation, as shown in dashed lines in FIG. 6, where connecting element 100 is parallel or generally parallel to extender 20a and then advanced distally along passage 30a and guided by tabs 26a, 28a to receiving portion 52a. Extender 20a can be pivoted relative to anchor 50a to a desired orientation, as indicated by arrowed lines 27a, either before or after connecting element 80 is inserted into extender 20a.

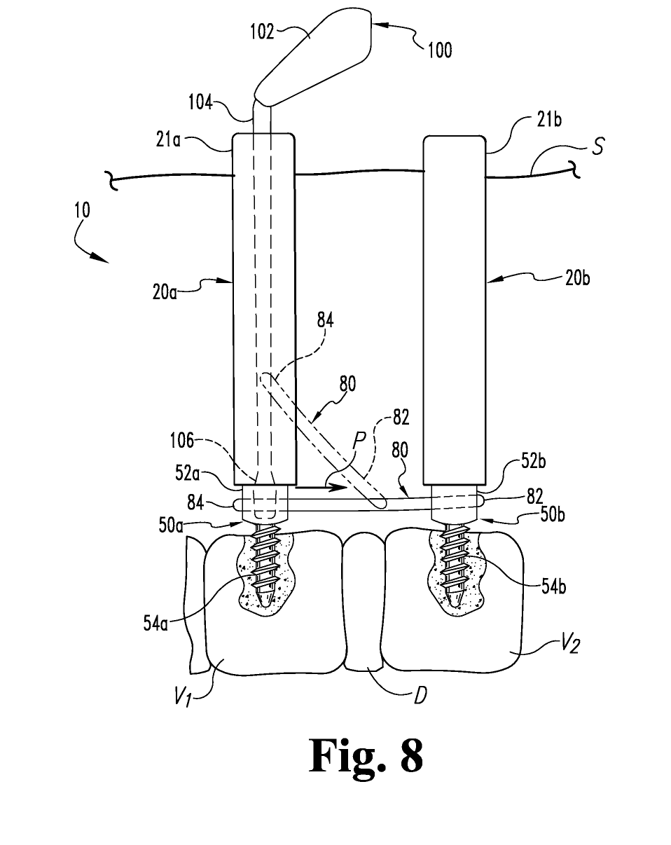

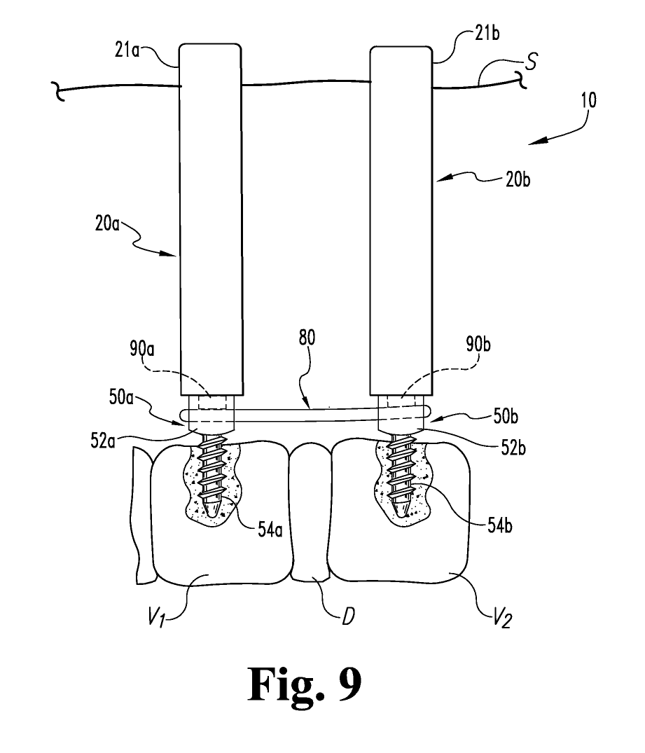

While connecting element 80 is held in the patient with inserter 100 either in extender 20a as shown in FIG. 7 or in a pivoted orientation as shown in FIG. 8, a second anchor 50b and extender 20b are inserted into the patient and engaged to vertebra V₂, as shown in FIG. 7. The prior insertion and orienting of connecting element 80 and extender 20a can provide an indication of a desirable implantation location for anchor 50b and extender 20b. For example, extender 20b can be pivoted universally about bone engaging portion 54b, such as indicated by arrowed lines 27b, to any parallel or non-parallel orientation relative to extender 20a. As shown in FIG. 8, when anchor 50b and extender 20b are positioned in the desired implantation location and orientation, first end portion 82 of connecting element 80 can be rotated and advanced subcutaneously toward receiving portion 52b of anchor 50b with inserter 100. First end 82 of connecting element 80 is movable with inserter 100 along insertion path P through tissue of the patient from a location outside the receiving portion 52b and then through receiving portion 52b so that connecting element 80 is located in each of the receiving portions 52a, 52b. As shown in FIG. 9, engaging members 90a, 90b can then be guided along extenders 20a, 20b and into receiving portions 52a, 52b to secure connecting element 80 therein and inserter 100 is removed from the patient. In the embodiment of FIGS. 6-9, no incision is provided between extenders 20a, 20b to accommodate connecting element 80 or inserter 100 as it guides connecting element 80 to its implantation location in receiving portions 52a, 52b.

In one embodiment, systems for positioning a connecting element adjacent the spinal column in minimally invasive surgical procedures include one or more extenders removably engaged to one or more anchors engaged to a bony segment. The anchor extenders provide a reference to the respective anchor locations within the patient even when the anchor is obstructed by skin and/or tissue of the patient. Similarly, the anchor extenders are sized such that a portion thereof extends above the skin of a patient when they are engaged to the bone anchors. In one form, it is contemplated that separate incisions may be made for using and positioning each anchor and anchor extender. An inserter instrument is engageable with a connecting element to move the connecting along a longitudinal axis of one of the anchor extenders and into the patient to a first location. A second anchor and extender can then be inserted into the patient and engaged to a second bony structure using the first extender and connecting element as a reference for the implantation location. The connecting element is then moved into the second anchor and secured to the first and second anchor extenders. Still, it should be appreciated that alternative forms, aspects, configurations, arrangements and methods are contemplated with respect to the subject matter disclosed and described herein.

In the illustrated forms of system 10, anchor extender 20a is configured the same as anchor extender 20b. However, in other forms, it is contemplated that anchor extender 20b could be configured differently than anchor extender 20a so long as it facilitates engagement of connecting element 80 to receiving portion 52b and anchor 50b to the bony structure. Additional features and embodiments of anchor extenders and inserters are provided in U.S. Pat. No. 6,530,929 issued Mar. 11, 2003; U.S. Pat. No. 7,188,626 issued Mar. 13, 2007; U.S. Pat. No. 7,465,306 issued on Dec. 16, 2008; U.S. Pat. No. 7,520,879 issued Apr. 21, 2009; U.S. Pat. No. 7,597,694 issued on Oct. 6, 2009; U.S. Patent App. Pub. No. 2005/0171540 published on Aug. 4, 2005; U.S. Patent App. Pub. No. 2007/0049931 published on Mar. 1, 2007; U.S. Patent App. Pub. No. 2008/0249531 published on Oct. 9, 2008; U.S. Patent App. Pub. No. 2008/0319477 published on Dec. 5, 2008; and U.S. Patent App. Pub. No. 2009/0264930 published on Oct. 22, 2009; each of which is incorporated herein by reference in its entirety.

In the illustrated embodiments, connecting element 80 is a rigid rod with an elongated body 86 extending between its leading end 82 and trailing end 84. Connecting element 80 can be linear along its length, or include a curvature defines by one or more arcs that extend along the length of connecting element 80. However, it is contemplated that connecting element 80 can have a curvature that varies or is compounded along its length, or could include linear and curved segments. In addition, in other forms it is contemplated that connecting element 80 can include any configuration known for a rod, implant, or fastener, so long as connecting element 80 is insertable into the patient and engageable to receiving portions 52a, 52b to guide its insertion in order to stabilize adjacent vertebrae V₁, V₂. Further, it is contemplated that connecting element 80 can be non-rigid, elastic and/or super-elastic and in the form of a cable, band, wire, or artificial ligament that is used in tethering, guiding, or other surgical procedures. The connecting element can include one or more acute bends and variable arcs. For non-rigid connecting elements, a rigid carrier can be provided to which connecting element 80 is mounted for insertion through anchor extender 20 as discussed further below.

It is contemplated that the connecting element can be comprised of a metal material, such as stainless steel, titanium, chrome-cobalt alloys. The connecting element may also be comprised of a polymer, such as, for example, polyetheretherketone (PEEK), polyetherketoneketone (PEKK), polymethylmethacrylate, polyurethane, silicone, silicone-polyurethane copolymers, epoxy, polycarbonate, polyketone, polyester, polyethylene, polyimide, polylactic acid, polypropylene, polystyrene, polysulfone, polyvinyl chloride, polyamide, poly(tetrafluoroethene), polyphthalamide, polybutylene and mixtures or combinations of thereof. In addition, the connecting element can be rigid to completely prevent spinal motion when secured to the anchors, semi-rigid to allow at least limited position, or flexible to permit motion between predefined limits of extension and/or flexion.

Alternative configurations of the systems described herein are also contemplated. For example, in one or more forms the systems described herein can be configured to insert a connecting element that extends across and is engaged to anchors positioned at three or more vertebral levels or to three or more bony portions or segments. In addition, use of the systems described herein for stabilization of bones, bony structures or other anatomical features besides vertebral stabilization are contemplated. Furthermore, the systems and instrumentation described herein may also be used in surgical procedures involving animals, or in demonstrations for training, education, marketing, sales and/or advertising purposes. In addition, the systems and instrumentation described herein may be also used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present application and is not intended to make the present application in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. A method for minimally invasive surgery, comprising:
engaging a first bone anchor to bony structure of a patient with a first extender extending proximally from the first bone anchor to a proximal end of the first extender located outside the patient;
positioning a connecting element into the patient by guiding the connecting element along the first extender to a first position adjacent the first bone anchor;
after positioning the connecting element into the patient, engaging a second bone anchor to bony structure of the patient with a second extender extending from the second bone anchor to a proximal end of the second extender located outside the patient; and
moving the connecting element from the first position to a second position where the connecting element extends between and connects the first and second bone anchors to one another.

2. The method of claim 1, wherein positioning the connecting element includes orienting the connecting element in a transverse relationship to the first extender so that the connecting element extends through opposite sides of the first extender.

3. The method of claim 2, wherein moving the connecting element from the first position to the second position includes moving the connecting element while maintaining the transverse relationship between the first extender and the connecting element.

4. The method of claim 2, further comprising engaging the connecting element to an inserter before positioning the connecting element so that the connecting element includes a first end having a length extending from the inserter and the length corresponds to a width of a receiving portion of the second anchor in which the connecting element is positioned.

5. The method of claim 1, wherein each of the first and second extenders includes first and second elongated tabs integrally formed with and removable from a proximal receiving portion of respective ones of the first and second bone anchors.

6. The method of claim 5, further comprising removing the first and second tabs of each of the first and second extenders after moving the connecting element into the proximal receiving portions of the first and second anchors.

7. The method of claim 1, wherein positioning the connecting element includes orienting the connecting element in a generally parallel relationship to the first extender so that the connecting element is located in and extends along a passage defined by the first extender when in the first position.

8. The method of claim 7, wherein moving the connecting element from the first position to the second position includes pivoting the connecting element so that a leading first end of the connecting element that is located adjacent a proximal receiving portion of the first bone anchor is moved toward the second bone anchor while an opposite trailing second end of the connecting element is moved toward the proximal receiving portion of the first bone anchor.

9. The method of claim 8, further comprising securing the connecting element to the first and second receiving portions with first and second engaging members engaged to respective ones of the first and second receiving portions.

10. A method for minimally invasive surgery, comprising:
engaging a first bone anchor to a first vertebra of a patient with a first extender extending proximally from the first bone anchor to a proximal end of the first extender located outside the patient;
positioning a connecting element into the patient by guiding the connecting element along the extender to a first position adjacent the first bone anchor; and
with the connecting element in the first position, referencing a second bone anchor to the first position of the connecting element to engage the second bone anchor to a second vertebra of the patient with a second extender extending from the second bone anchor to a proximal end of the second extender located outside the patient.

11. The method of claim 10, further comprising:
moving the connecting element in the patient from the first position to a second position where the connecting element extends between and connects the first and second bone anchors to one another.

12. The method of claim 11, wherein positioning the connecting element includes orienting the connecting element in a transverse relationship to the first extender so that the connecting element extends from opposite sides of the first extender.

13. The method of claim 12, wherein moving the connecting element from the first position to the second position includes moving the connecting element while maintaining the transverse relationship between the first extender and the connecting element and further comprising:
referencing a third bone anchor to the connecting element when the connecting element is moved to the second position to engage the third bone anchor to a third vertebra of the patient with a third extender extending from the third bone anchor to a proximal end of the third extender located outside the patient; and
moving the connecting element from the second position to a third position where the connecting element extends between and connects the first, second and third bone anchors to one another.

14. The method of claim 12, further comprising engaging the connecting element to an inserter before positioning the connecting element so that the connecting element includes a first end having a length extending from the inserter and the length corresponds to a width of a receiving portion of the second anchor in which the connecting element is positioned.

15. The system of claim 12, wherein each of the first and second extenders includes elongated first and second tabs extending from a proximal receiving portion of a respective one of the first and second bone anchors, the first and second tabs defining a passage therebetween that opens along opposite sides of a corresponding one of the first and second extenders and the passage further opening at a proximal end of the corresponding one of the first and second extenders, and positioning the connecting element includes positioning the connecting element in the passage of the first extender so that the connecting element extends from the opposite sides of the first extender.

16. The method of claim 15, further comprising removing the first and second tabs of each of the first and second extenders after moving the connecting element into the proximal receiving portions of the first and second anchors.

17. The method of claim 10, wherein positioning the connecting element includes orienting the connecting element in a generally parallel relationship to the first extender so that the connecting element is located in and moves along a passage defined by the first extender as the connecting element is positioned in the patient.

18. The method of claim 17, wherein positioning the connecting element in the first position includes pivoting the connecting element from the first extender so that a leading first end of the connecting element is moved toward the second bone anchor while an opposite trailing second end of the connecting element is moved toward the proximal receiving portion of the first bone anchor.

19. The method of claim 18, further comprising:
moving the connecting element in the patient from the first position to a second position where the connecting element extends between and connects the first and second receiving portions of the first and second bone anchors to one another; and
securing the connecting element to the first and second receiving portions with first and second engaging members engaged to respective ones of the first and second receiving portions of the first and second bone anchors.

20. The method of claim 19, wherein the first and second receiving portions are pivotal relative to first and second bone engaging portions of the first and second bone anchors.
